(19) United States
(12) Patent Application Publication
(10) Pub. No.: US 2021/0330927 A1
(43) Pub. Date: Oct. 28, 2021

(54) METHOD OF PERFORMING CRYONEUROLYSIS

(71) Applicant: Black Cat Medical LLC, Fortville, IN (US)

(72) Inventor: Michael F. Guzman, Fortville, IN (US)

(73) Assignee: Black Cat Medical LLC, Fortville, IN (US)

(21) Appl. No.: 17/237,146

(22) Filed: Apr. 22, 2021

Related U.S. Application Data
(60) Provisional application No. 63/015,864, filed on Apr. 27, 2020.

(51) Int. Cl. A61M 19/00; A61F 7/12

(52) U.S. Cl. CPC A61M 19/00; A61B 17/3403; A61F 2007/126; A61F 7/12

(57) ABSTRACT

Provided are methods for anesthetizing a human patient undergoing surgery, and/or pain block procedures. In some forms, the methods include inserting at least one cryo-needle into a target region of the patient, the target region including a site superficial to a target intercostal nerve and cooling the cryo-needle to inhibit the target intercostal nerve.

### REFERENCE TO RELATED CASES

This application claims the benefit of U.S. Provisional Patent Application No. 63/015,864, filed Apr. 27, 2020, which is incorporated by reference herein in its entirety.

### BACKGROUND

The present disclosure relates to pain reduction associated with intercostal nerves of the human body.

This present disclosure relates to targeted blockade of nerve impulses within the intercostal nerves. Target nerves for chest procedures are called intercostal nerves. Intercostal nerve blocks were initially described around 1905. This block was not widely utilized for numerous reasons. For example, the intercostal nerves are in close proximity to the lung and fear of a pneumothorax is well known. Local anesthetics may limit the duration of sensory block only for 6-12 hours. Advances with ultrasound and continuous catheters may extend the pain relief for 24-48 hours however such a procedure requires intervention of a highly skilled anesthesioligust, which may not always be available.

The present disclosure provides an improved method for blocking pain associated with intercostal nerves for extended periods.

### SUMMARY

In certain aspects, the present disclosure pertains to systems and methods for administering anesthesia and/or pain blocks to a patient undergoing surgery. In accordance with some forms, the present disclosure provides methods of performing cryoneurolysis to anesthetize patient tissue. Accordingly, in one embodiment the present disclosure provides a method for anesthetizing a human patient undergoing surgery, and/or pain block procedures, the method comprising: inserting at least one cryo-needle into a target region of the patient, the target region including a site superficial to a target intercostal nerve, the cryo-needle having a distal end configured to cool surrounding patient tissue, and cooling the distal end of the cryo-needle to to cause cooling of surrounding patient tissue thus inhibiting one or more intercostal nerves in the surrounding patient tissue. In certain embodiments, the target region may be sterilized prior to inserting the cryo-needle into patient tissue. In some forms, the cryo-needle is imageable such that placement of the cryo-needle can be assisted and/or confirmed using a medical imaging system. In accordance with certain inventive variants, the methods may also include administering a local anesthetic to the target region, for example liposomal bupivacaine.

In certain embodiments, the present disclosure provides a method for anesthetizing a human patient undergoing a mastectomy procedure. Thus in some forms, the target region comprises a posterior mid axillary region of the patient spanning T2-T7. In some forms, the mastectomy procedure may comprise a bilateral mastectomy. In certain embodiments, the mastectomy procedure includes reconstruction of the breast.

In certain embodiments, the present disclosure provides a method for anesthetizing a human patient undergoing a hernia repair procedure. In some forms, the hernia repair procedure comprises inguinal hernia repair surgery. Thus in some forms, the target region comprises a generally linear region between the patient's anterior iliac spine and the patient's umbilicus. In accordance with certain inventive methods, the target region is subject to at least 3, for example 5, cooling cycles, each including inserting the cryo-needle within the target region and cooling patient tissue at the insertion site.

Additional embodiments, as well as features and advantages of embodiments of the invention will be apparent from the description herein.

### BRIEF DESCRIPTION OF THE DRAWINGS

FIG. 1 is one embodiment of a method as disclosed herein.

FIG. 2 is a diagram of one embodiment of a method as disclosed herein.

### DESCRIPTION OF THE SELECTED EMBODIMENTS

For the purpose of promoting an understanding of the principles of the disclosed technique, reference will now be made to the embodiments illustrated in the figures and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail; although it will be apparent to those skilled in the relevant art that some features that are not relevant to the present invention may not be shown for the sake of clarity.

As used here (claims, specification, and other definitions) the following terms have the following meaning:

Articles and phrases such as, “the”, “a”, “an”, “at least one”, and “a first”, “comprising”, “having” and “including” here are not limited to mean only one, but rather are inclusive and open ended to also include, optionally, two or more of such elements and/or other elements. In terms of the meaning of words or terms or phrases herein, literal differences therein are not superfluous and have different meaning, and are not to be synonymous with words or terms or phrases in the same or other claims.

The term “means for” in a claim invokes 35 U.S.C. § 112(f), literally encompassing the recited function and corresponding structure and equivalents thereto. Its absence does not, unless there otherwise is insufficient structure recited for that claim element. Nothing herein or elsewhere restricts the doctrine of equivalents available to the patentee.

The term “and/or” is inclusive here, meaning “and” as well as “or”. For example, “P and/or Q” encompasses, P, Q, and P with Q; and, such “P and/or Q” may include other elements as well.

The term “anesthetizing” as used herein has the meaning, a process for providing temporary loss of sensation, muscle control, and/or awareness.

The term “anterior” as used herein has the meaning, being situated nearest or toward the front of the body. With respect to the spine, the anterior is considered to be the side of the spine closest to the stomach or the throat.

The term “anteriorly” as used herein has the meaning, positioned in a location more anterior with respect to another object where the anterior refers to a position nearer to the front of a reference point.

The term “body portion” as used herein has the meaning, the main or principal part of an object. More specifically, it refers to the part of a vertebra implant that contacts the vertebral body of a neighboring vertebra.

The term “contact” as used herein has the meaning, of two objects the state or condition of physical touching. As used, contact requires at least one location where objects are directly or indirectly touching, with or without any other member(s) material in between.

The term “cranial-caudal orientation” as used herein has the meaning, in the direction along an axis running vertically toward and away from a (standing) patient's skull.

The term “cranial direction” as used herein has the meaning, in the direction toward the patient's head along the cranial-caudal axis.

The term “caudal direction” as used herein has the meaning, in the direction towards the patient's feet along the cranial-caudal axis.

The term “cryoanalgesia” (also known as cryoneurolysis) as used herein has the meaning is the reversible destruction of the signal carrying parts of a nerve. It is a small subset of treatments under the broad umbrella of cryotherapy.

The term “cryo-needle” as used herein has the meaning, a medical device having a slender, usually sharp pointed body and configured to cause freezing at the distal end, (which may include its tip, but also may be portions near to but proximal to the tip), upon activation.

The term “facilitate” as used herein has the meaning, to aid or help accomplish an action or a process to make that action or process easier. The act of facilitation does not need to accomplish the action or process entirely on its own.

The term “front surface” as used herein has the meaning, an exterior surface that may or may not be the anterior most surface.

The term “hole” as used herein has the meaning, a hollow opening within a body, structure, or an object. It can be any shape.

The term “imageable” as used herein has the meaning, a device that is configured to allow for medical imaging of the device during use such as by computed tomography (CT), magnetic resonance (MRI), positron emission tomography (PET), fluoroscopy, and/or ultrasonography. As used herein the “imageable” device is configured to allow a user to visualize all or a portion of the device within the patient during a procedure.

The term “pain block” as used herein has the meaning, inhibition of one or more sensory nerves that relay pain signals to a target region of the patient's body.

The term “intercostal nerve” as used herein has the meaning, the anterior rami of the first 11 thoracic spinal nerves, which are positioned within the intercostal space. Intercostal nerves are peripheral nerves. Each intercostal nerve enters the corresponding intercostal space between the posterior intercostal membrane and the parietal pleura. The nerve then travels forward with the intercostal vessels in the costal groove of the corresponding rib, between the internal and innermost intercostal muscles. The first thoracic nerve divides into a superior part, which joins the brachial plexus, and an inferior part, which becomes the first intercostal nerve. The first six intercostal nerves innervate nearby intercostal muscles, the cutaneous territory on the side of the thoracic wall, the thoracic pleura, and end as terminal branches (anterior cutaneous branch) to innervate the skin near the midline of the chest. The lower intercostal nerves leave their intercostal spaces anteriorly (after giving branches to the intercostal muscles, abdominal peritoneum, and skin) to innervate the anterior abdominal wall, muscles (in particular the rectus abdominis), and the overlying skin. Near their origin, the intercostal nerves send a posterior branch to the paraspinal muscles and the overlying skin, and communicant rami to the sympathetic trunk adjacent to the vertebral column.

The term “local anesthesia” as used herein has the meaning, a medication that causes reversible absence of pain sensation in a target area of the body. When it is used on specific nerve pathways, paralysis also can be achieved.

The term “sensory nerve” as used herein has the meaning, a nerve that carries sensory information toward the central nervous system (CNS).

The term “side” as used herein has the meaning, one of the faces on the surface of an object. An object can have multiple faces with a variety of orientations. For example, an object may have a front side, a bottom side, a back side, or a top side.

The term “superficial” as used herein has the meaning, a directional term that indicated one structure is located more externally than another, or closer to the surface of the body.

The term “transverse” as used herein has the meaning, situated or extending across an object or an axis.

The term “withdrawal” as used herein has the meaning, the action of removing or taking away something for a particular location.

With cryo-needle systems, doctors are able to deliver precise, controlled doses of cold temperature only to the targeted nerve through a handheld device. This needle-based procedure is safe, and does not damage or destroy the surrounding tissue. Cryo-needle based treatments use the body's natural response to cold to treat peripheral nerves and immediately reduce pain—without the use of drugs. Treated nerves are temporarily stopped from sending pain signals for a period of time, followed by a restoration of function.

Cryo-needle therapies, work by applying targeted cold to a peripheral nerve, such as an intercostal nerve. A precise cold zone is formed under the skin - cold enough to immediately prevent the nerve from sending pain signals without causing damage to surrounding structures. The effect on the nerve is temporary, providing pain relief until the nerve regenerates and function is restored.

Cryoneurolysis procedures can include surgeries of the anterior and posterior chest, with sensory pain relief lasting weeks. In accordance with some modes of practicing the disclosed methods, patients are placed in a lateral decubitus, surgical site up position. In certain embodiments, an intercostal nerve block with local anesthetic is performed before cryoneurolysis. This is done to prevent discomfort that is often associated with cryoneurolysis therapy.

In certain embodiments, cryoneurolysis is performed with a single tip cryoneurolysis needle. Placement of the cryoneedle may comprise ultrasound guidance as disclosed herein. In some forms, cryoneurolysis needles are dull and difficult to penetrate the fascial planes of the serratus anterior and the outer intercostal membrane. In certain embodiments, a multipronged cryoneurolysis needle facilitates placement. In certain embodiments, a 3-prong linear array cryoneurolysis needle is used. Such multi-prong linear needle arrays significantly remove any risk of pneumothorax because the needle length is not likely long enough to reach the pleura. The supported mechanism of action for cryoneurolysis with a multi-prong needle is extension of the cryoneurolysis freeze ball into the intercostal nerve plexus in the serratus anterior plane or possibly below the outer intercostal membrane. Cryoneurolysis with a single long needle requires 1-2 freeze cycles/side at each targeted intercostal space. Cryoneurolysis with a 3-prong needle may require 14-16 freeze cycles per side. In accordance with some forms, a target region is subjected to at least three freeze cycles, preferably 3-20 freeze cycles. In some forms, the target region is subject to five freeze cycles. As used herein a “freeze cycle” is synonymous with “cryoneurolysis cycle” and comprises insertion and cooling of a cryo-needle into patient tissue at a distinct site within a target area.

In certain embodiments the present disclosure provides a method for anesthetizing a human patient undergoing surgery, and/or pain block procedures, the method comprising: sterilizing the patient's skin including a target region, the target region including a site superficial to a target intercostal nerve; inserting at least one cryo-needle into a first tissue region at or near the target site, the cryo-needle having a distal end configured to cool surrounding patient tissue; cooling the distal end of the cryo-needle to cause cooling of surrounding patient tissue thus inhibiting one or more intercostal nerves in the surrounding patient tissue; thereafter, performing surgery on the patient.

In accordance some forms of practicing the disclosed methods, an imageable cryo-needle is used, such that the cryo-needle may be placed using guidance from a medical imaging device.

In certain embodiments, the cryo-needle comprises two or more needle ends configured to cool surrounding patient tissue. In some forms, the two or more needle ends are configured in a generally linear array.

In some forms, the present disclosure provides a method of performing cryoneurolysis for mastectomy. In accordance with certain embodiments, a local anesthetic, such as bupivacaine, is placed in conjunction with the cryoneurolysis procedure. Ultrasound guided liposomal bupivacaine infiltration into specific intercostal spaces can provide relief for 36-48 hours and avoid pain associated with drains, catheters, and pumps. Breast surgeons typically leave drains in place for 7-10 days. Many patients report that the pain and irritation associated with the drain is worse than the mastectomy procedure itself.

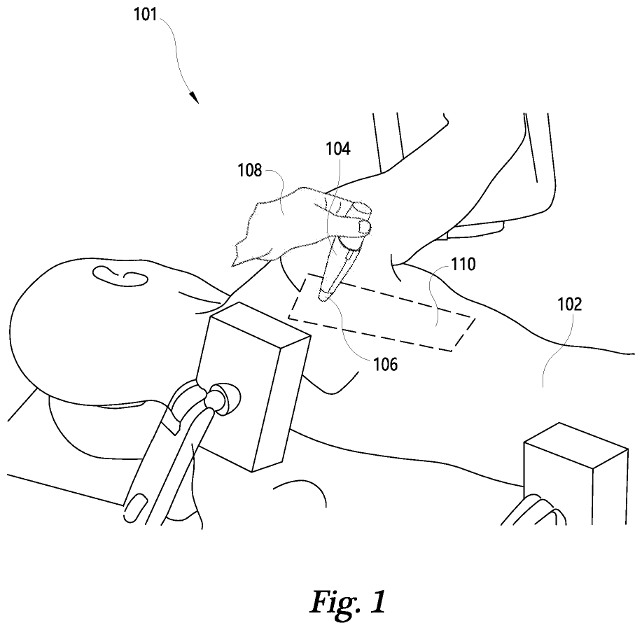

Turning now to a discussion of the illustrated embodiments, FIG. 1 shows one embodiment of a method 101 for anesthetizing a patent. In the illustrated embodiment, the patient 102 is lying in the lateral decubitus position. Target region 110 is identified for cryoneurolysis. In the illustrated embodiment, the target region comprises a posterior mid axillary region extending from about T1 to T7, preferably T2-T6. In the illustrated embodiment, a single target region is identified, for example for a unilateral mastectomy procedure. As discussed herein it is within the scope of the present disclosure to provide bilateral target regions for bilateral anesthetization, for example for a bilateral mastectomy. An operator 108, for example a surgeon or anesthesiologist, places cryoneurolysis device 104, having a distal end 106 having one or more needles within the target region, and performing one or more cryoneurolysis cycle each comprising inserting the cryo-needle into patient tissue and cooling the distal tip of the cryo-needle.

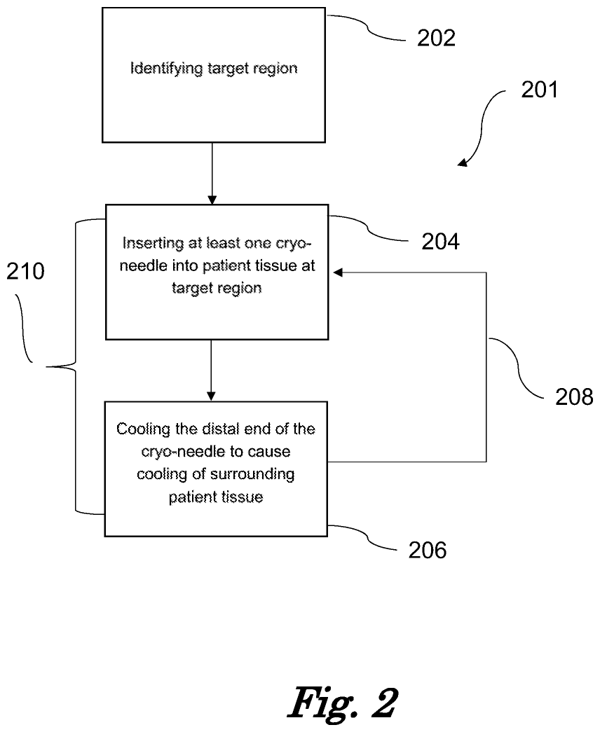

FIG. 2 illustrates ne embodiment of a method 201 for anesthetizing a patient as disclosed here. As detailed herein, method of the current disclosure may include further steps, for example administration of a local anesthetic and/or monitored anesthesia care. The illustrated embodiment includes the step of identifying a target region 202. Such target region is preferable associated with one or more intercostal nerves. Exemplary target regions as disclosed herein include a posterior mid axillary region at around T1-T8, preferably T2-T6, and or extending from the patient's iliac spine to umbilicus. Additional intercostal nerves may be target along their respective lengths at other target regions. The illustrated method includes one or more cryoneurolysis cycles 210. Each cycle includes the step of inserting at least one cryo-needle into patient tissue at the target region 204, and cooling the distal end of the cryo-needle to cause cooling of surrounding patient tissue 206. The cooling cycle 210 may be repeated 208 as needed to effect sufficient cooling of the underlying target nerve.

It is within the scope of the present disclosure to provide systems and methods for training the disclosed procedures. For example, in some forms a medical training phantom may be provided having one or more of the intercostal nerves and related anatomy as described herein. It is within the scope of the present disclosure to practice the methods described herein with a training phantom or other analogous training implement. It is also within the scope of the present disclosure to provide training material relating to the performance of the surgical techniques described herein. Such training materials may be in any suitable form, for example written, verbal, or recorded instructing.

For the purposes of promoting further understanding of aspects of the present disclosure and their features and advantages, the following specific examples are provided. It will be understood that these examples are illustrative, and not limiting, or embodiments of the present disclosure.

### EXAMPLES

### Example 1

A 55 year-old female underwent a single sided mastectomy and axillary node dissection for breast cancer. There was no breast reconstruction.

Preoperatively, the patient was sedated with midazolam 2 mg IV and received acetaminophen 1000 mg IV. The patient was monitored and prepared using sterile techniques. A mixture of Liposomal bupivacaine 133 mg (10 ml), bupivacaine 75 mg (30 ml), and normal saline (60 ml) was prepared totaling 100 ml). A 4-inch ultrasound needle was used to infiltration of the mixture into the intercostal spaces. The patient received a general anesthetic with propofol and sevoflurane. The patient was given ketorolac 15 mg every 6 hours and one additional dose of acetaminophen 1000 mg intravenously.

Results. The patient emerged from anesthesia with a pain score of 0/10. The patient did not request any additional pain medications for her 20-hour outpatient surgery stay. The patient was contacted after her surgery and reported the only pain medicines she had taken for 3 weeks was two acetaminophen 500 mg tablets.

Discussion. Remarkable postoperative pain control synergy is possible when combining infiltration of liposomal bupivacaine and percutaneous cryoneurolysis for mastectomy patients.

### Example 2—Intercostal Liposomal Bupivacaine and Percutaneous Cryoneurolysis Synergy For Mastectomy and Breast Reconstruction

Breast cancer is the most common cancer in women, excluding skin cancers. Approximately one in eight women develop breast cancer in their lifetime. An estimated 281,000 new 2021 cases of invasive cancer are expected in the USA. Multiple successful short duration (24-36 hour) pre-op pain block techniques have been documented for postoperative discomfort after mastectomy with or without reconstruction. 20-50% of women post mastectomy develop post mastectomy pain syndrome (PMPS). PMPS symptoms are persistent chest, axilla, arm, and shoulder pain lasting more than three months after surgery.

Sixty-four patients were scheduled for mastectomy, unilateral or bilateral, with or without reconstruction. Three of the sixty-four patients were male. Patients were placed in a lateral decubitus position. Sedation for the blocks included midazolam 2 mg, and ketamine 5 mg. The patients received intercostal infiltration pain blocks using liposomal bupivacaine (LB) expanded in normal saline (NS). Unilateral procedures had LB (266 mg/20 ml) expanded in NS 55 ml, for a total Of 75 ml of LB dilution used for intercostal infiltration pain blocks T1-T7. Bilateral procedures had LB (266 mg/20 ml) expanded in NS 130 ml, for a total of 150 ml of LB dilution (75 ml/side) used for intercostal infiltration pain blocks T1-T7 bilaterally. In addition, patients received unilateral or bilateral percutaneous cryoneurolysis using a three-prong needle in a posterior mid axillary line from T2-T6. Patients received propofol and sevoflurane general anesthetic. Fentanyl 50-100 mcg was given on induction of general anesthesia based on anesthesia provider preference. The patients received IV acetaminophen 1000 mg every 12 hours and ketolorac 15 mg every 6 hours.

95% of the patients were scheduled in our surgery center for a 24-hour length of stay. 100% of the patients had pain scores of 0-2/10 within 4-hours of emergence from surgery. 100% of the patients had pain scores of 0-1/10 on discharge. 63/64 patients had prolonged sensory pain relief after hospital discharge lasting 1-6 weeks. The single patient without prolonged sensory pain relief was a male post unilateral mastectomy. This patient required zero opioid pain medications during hospitalization.

Discussion. Breast surgeons typically leave drains between surgical planes for seven to ten days to avoid hematoma complications. Many women complain that the discomfort from the hematoma drains was worse than their mastectomy. 20-50% of women post mastectomy develop post mastectomy pain syndrome (PMPS). Intercostal infiltration of liposomal bupivacaine and percutaneous cryoneurolysis has unsurpassed, immediate postoperative pain relief synergy for mastectomy and reconstruction. This combination pain technique could reduce the incidence of PMPS.

### Example 3—Synergistic Effect of Liposomal Bupivacaine and Percutaneous Cryoneurolysis for Lower Abdominal Surgery

Inguinal hernia is one of the more common general surgery procedures in the United States. The optimal approach has been debated for more than 20 years. Operative techniques comparing open, laparoscopic transabdominal, total extraperitoneal, and robotic preperitoneal repair have all advocated advantages. These innovative minimal invasive techniques have reported less pain, including both acute and persistent chronic discomfort. Laparoscopic and robotic proponents also believe patients experience shorter length of hospital stay, and earlier return to work.

Open inguinal hernia repair with mesh was performed on an 81-year-old male patient. Monitored Anesthesia Care (MAC) was used. Liposomal bupivacaine 133 mg (10 ml) was diluted with bupivacaine 0.25% (10 ml) and normal saline 80 ml. Total local dilution volume was 100 ml. 20 ml of the local dilution was given to the surgeon for skin infiltration. A distal TAP block was placed by ultrasound and 40 ml of local dilution was placed. The typical incision site for an open hernia repair is at the midpoint distance between the anterior iliac spine and pubic rami. Ultrasound anatomy at this midpoint will show the roof of the inguinal canal (external oblique). 20 ml of local dilution was placed at this midpoint below the external oblique and an additional 20 ml was infiltrated into surrounding subcutaneous tissue. The ultrasound probe was then placed in a transverse orientation to visualize the anterior iliac spin and a marker was drawn along the transducer pointing at the umbilicus. Percutaneous cryoneurolysis using a three-prong needle was performed for five cryoneurolysis cycles (one minute per cycle) along the marked line. The patient's surgery was performed with MAC anesthesia including nasal cannula oxygen, routine monitoring, midazolam 2 mg, and propofol 25 mg times two doses prophylactically for skin incision.

The patient coherently talked throughout surgery and never complained of any surgical discomfort. Zero pain medications were requested during hospitalization. Lower extremity motor strength was intact immediately after surgery. The patient was kept overnight in our surgery center for observation because of his severe COPD and recent Covid episode. A family member on a postoperative survey reported pain medications were never requested even at three weeks post-surgery.

Discussion. Operative time is obviously longer in laparoscopic and robotic surgery techniques. Complications with non-open techniques include vascular and bladder injury depending on skill training of the surgeon. Robotics have significant disadvantages including startup, maintenance, and instrumentation costs. Robotic surgery costs are reported 38% greater ($5000) than open and are prohibitive for institutions with declining reimbursement. A global review of 51,000 patients suggests laparoscopic or robotic inguinal hernia repair may have better outcomes in the short term. TAP blocks can reduce postoperative lower abdominal surgery pain, however surgical analgesia is unlikely. Addition of local anesthetic into the inguinal canal increases likelihood of an excellent sensory blockade. Percutaneous cryoneurolysis of the superficial ilioinguinal nerve medial to the anterior iliac crest resulted in sensory pain relief lasting weeks after surgery. Anesthesiologists can now provide a superior outcome for open hernia repair compared to minimal invasive techniques by combining liposomal bupivacaine TAP and inguinal canal blocks with percutaneous cryoneurolysis of the superficial ilioinguinal nerve. This pain technique combination has significant applications for extended postoperative pain relief for numerous lower abdominal surgery procedures.

The language used in the claims and the written description and in the above definitions is to only have its plain and ordinary meaning, except for terms explicitly defined above. Such plain and ordinary meaning is defined here as inclusive of all consistent dictionary definitions from the most recently published (on the filing date of this document) general purpose Webster's dictionaries and Random House dictionaries.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

What is claimed is:

1. A method for anesthetizing a human patient undergoing surgery, and/or pain block procedures, the method comprising:
inserting at least one cryo-needle into a target region of the patient, the target region including a site superficial to a target intercostal nerve, the cryo-needle having a distal end configured to cool surrounding patient tissue; and
cooling the distal end of the cryo-needle to cause cooling of surrounding patient tissue thus inhibiting one or more intercostal nerves in the surrounding patient tissue.

2. The method of claim 1, also comprising sterilizing the target region prior to said inserting.

3. The method of claim 1, wherein the cryo-needle is imageable.

4. The method of claim 1, comprising:
imaging the distal tip of the cryo-needle with a medical imaging device to confirm placement near the intercostal nerve.

5. The method of claim 1, wherein the cryo-needle comprises two or more needle ends configured in a generally linear array.

6. The method of claim 1, wherein the cryo-needle comprises three needle ends configured in a generally linear array.

7. The method of claim 1, also comprising:
administering a local anesthetic to the target region.

8. The method of claim 7, wherein the local anesthetic comprises liposomal bupivacaine.

9. The method of claim 1, wherein the surgery comprises a mastectomy procedure and wherein the target region is located at the posterior mid axillary region of the patient, spanning at least T2 to T7.

10. The method of claim 9, wherein the mastectomy procedure includes reconstruction.

11. The method of claim 9, wherein the mastectomy procedure comprises a bilateral mastectomy procedure, and wherein a target region is present bilaterally on the patient.

12. The method of claim 1, wherein the surgery comprises hernia repair surgery.

13. The method of claim 12, wherein the hernia repair surgery comprises inguinal hernia repair surgery.

14. The method of claim 13, wherein the target region is locates in a generally linear region between the patient's anterior iliac spine and the patient's umbilicus.

15. The method of claim 14, wherein at least 3 cryoneurolysis cycles, each including inserting the cryo-needle and cooling patient tissue at the insertion site, are performed along the target region.

16. The method of claim 15, wherein 5 cryoneurolysis cycles, each including inserting the cryo-needle and cooling patient tissue at the insertion site, are performed along the target region.
